(19) United States
(12) Patent Application Publication
(10) Pub. No.: US 2012/0004615 A1
(43) Pub. Date: Jan. 5, 2012

(54) Bandage

(75) Inventors: Andreas Schmidt, Duderstadt (DE); Jens Söder, Gottingen (DE)

(73) Assignee: MCAIRLAID'S VLIESSTOFFE GMBH & CO. KG, Berlingerode (DE)

(21) Appl. No.: 13/172,887

(22) Filed: Jun. 30, 2011

(30) Foreign Application Priority Data
Jul. 2, 2010 (DE) 20 2010 007 967.5

(51) Int. Cl. A61M 25/02

(52) U.S. Cl. 604/174

(57) ABSTRACT

A bandage for covering a body opening from which a conduit, not belonging to the body, projects, has a first support with a first slit beginning at an edge of the support and extending toward a center of the first support, wherein the first slit defines a first support half and a second support half on opposed sides of the first slit. A first flat absorbent core is disposed on the first support half; a second flat absorbent core is disposed on the second support half. The first and second absorbent cores are positioned in parallel orientation relative to the first slit, respectively. The first slit has an end remote from the edge of the first support; the end of the first slit is provided with a widened portion.

### BACKGROUND OF THE INVENTION

The present invention concerns a bandage for covering a body opening from which a conduit projects, the bandage comprising a support with a slit forming a first support half and a second support half wherein on each one of the two support halves, in parallel orientation to the slit, a flat absorbent core is disposed.

In emergency medicine, or for use in surgical procedures, bandages and, in particular, compresses have been known for a long time for treatment of acute wounds and surgically applied body openings from which blood, ichor, and other body liquids exude. These bandages have the purpose to cover the wounds or the body openings in order to prevent penetration of foreign matter into the wound and to absorb blood and ichor.

Slitted compresses are often used for covering body openings that have been surgically applied in order to drain body fluids through a cannula or a conduit. These slitted compresses are comprised in general of a rectangular nonwoven or other absorbent material that has a slit extending approximately to the center of the material. Because of the slit, such compresses have the advantage that application and removal or exchange of the compress is possible without the cannula or conduit having to be removed. The prior art discloses various protective compresses. For example, there are single-piece compresses that are comprised exclusively of absorbent material. These compresses have the disadvantage that, as a result of the relatively thick absorbent material, they are very rigid which is a disadvantage for the patient with respect to wearing comfort. Also, the material consumption of absorbent nonwoven is high which increases the material costs for such bandages.

In WO 2007/118664 a bandage (slitted compress) for placement about a surgically applied conduit at a body opening or body cavity is disdosed that has at least one liquid-absorbing mat with a slit wherein the liquid-absorbing mat is enclosed by an envelope that forms a barrier for solid coarse secretions and enables uptake or absorption of exuding substances and/or substances that must be removed by means of the absorbent body arranged within the cavity. The mat is moreover provided with at least one underlying foam layer.

The compresses of the prior art have the disadvantage that the absorbent material is formed as a single piece so that the compress itself is very rigid and the wearing comfort is not optimal. Moreover, the material consumption of absorbent material is very high. The slit has the disadvantage that cannulas or conduits of a larger cross-section are not optimally enclosed; bulges are formed so that the compress is not resting completely flat on the surface to be covered.

It is therefore an object of the present invention to provide a bandage, in particular a slitted compress, that does not have the disadvantages known from the prior art; in particular, the wearing comfort for the patient should be improved and the material costs should be reduced.

### SUMMARY OF THE INVENTION

The object of the present invention is therefore a bandage for covering a body opening from which a line, hose, conduit or the like, not belonging to the body, projects, which bandage has a support with a slit that begins at the edge of the support and extends into the support with formation of a first support half on one side and a second support half on the other side of the slit, wherein each one of the two support halves, in parallel orientation to the slit, has disposed thereon a flat absorbent core, characterized in that the slit in the area of its end is provided with a widened portion (expansion).

In the context of the present invention, the device such as a line, hose, conduit, cannula and the like, not belong to the body and projecting from the body opening, is referred to as conduit.

The bandage according to the present invention has the advantage that it can be placed for covering wounds or surgically applied body openings also about conduits and the like, not belonging to the body, without an exchange of these conduits being required. Also, an exchange of the bandage is possible without the conduit not belonging to the body having to be exchanged. This conduit is enclosed by the widened portion in the area of the end of the slit; the edges of the slit are thus resting also substantially flat on the body surface to be bandaged. A further advantage is that the actual absorbent material, a flat absorbent core, that is to absorb the bodily liquid is applied on one support in the form of two material sections that are separated from each other. The support material on an extension of the slit has in general no absorbent core so that the bandage, as a whole, is more flexible; this leads to increased comfort for the patient.

It has been found to be suitable that the widened portion is located in the area of the slit end and is of greater width than the slit. The size of this opening should be matched to the conventionally used conduits for draining bodily liquids as they are used in surgery and in patient care. For designing the widened portion in the area of the slit end there is a variety of different design possibilities available. The widened portion can be designed, for example, like a keyhole expansion that may be round, triangular, quadrangular or also polygonal. In a possible further embodiment the slit end is designed in the form of a cross. In a further possible embodiment, the slit end is provided with a widened portion extending transversely to the slit so that the slit has the shape of a “T”; the widened portion extending transversely can be straight or can be of an arc shape.

The absorbent core or the absorbent cores according to the invention are applied to a support. Usually, the bandage is resting with the support side on the body surface of the patient. The support material should be designed such that it prevents penetration of foreign matter from the exterior into the opening and that it has such a structure that the bodily liquid, via the support material, can diffuse into the absorbent core and can be absorbed by the absorbent core. As materials for the support, in particular nonwovens (fleece) and woven materials of natural or synthetic fibers and/or punctured two-dimensional and/or three-dimensional films have been found suitable. These materials are known to a person of skill in the art from the field of wound care.

In a preferred embodiment, the flat absorbent core has an additional support also on the flat side that is facing away from the support. This additional support has preferably the same shape as the first support. One or both supports extend preferably past the surface of the absorbent core and are connected at their projecting portions with each other so that the flat absorbent core is essentially enveloped by the two support layers. The two support layers are preferably adhesively connected to each other at their projecting portions but they can also be fused or can be connected in another known suitable waywith each other. The first support and the additional support can be comprised of the same or different materials; preferably, both supports are comprised of nonwoven. The nonwoven (fleece) has the advantage that exiting bodily liquid can be transported well from the body opening to the absorbent core. The support material that is facing the body surface can also be coated, outside of the area that is to cover the body opening, entirely or partially with an adhesive agent so that the bandage, without additional aid, can be attached or positionally fixed to the body surface.

In a possible embodiment of the present invention, the flat absorbent cores are covered on opposite flat faces or sides by a support material with slit. Preferably, in areas in which the two supports directly abut each other, the support material of the two supports is connected to each other by an adhesive or in another way. This connection has in particular the advantage that the exiting body liquid can be transported th rough both support layers to the absorbent core. The absorption performance of the bandage according to the invention can be further increased when the absorbent core on its flat side is directly connected with the support orthe supports, for example, adhesively. By this connection, a particularly fast absorption of the bodily liquid by means of the absorbent core is provided; the flow of the absorbed liquid is not interrupted by an intermediate space between support and absorbent core. Moreover, with this connection the stability of the bandage according to the invention is increased.

The flat absorbent core can be of any absorbent material such as cellulose and/or another material with high absorption capacity. The absorbent core serves for taking up the exudates exiting from the body opening and to absorb them permanently. Rewetting in the direction toward the body opening should be advantageously prevented as much as possible. The absorbent core is therefore preferably formed of a material which is capable of not only absorbing the bodily liquids but also to store them, such as pulp or pulp-related materials as well as synthetic absorbent materials. The absorbent core is preferably a nonwoven of pulp fibers such as an airlaid material.

Preferably, an airlaid product of pulp fibers is used as an absorbent material. In a preferred embodiment of the present invention, the absorbent core has distributed across its surface area embossed areas in which the fibers, preferably pulp fibers, are compressed with each other more strongly than in other areas and in this way are connected to each other as a result of the compression forces without adhesives and/or binding agents.

In one embodiment the absorbent core is produced from a fiber web of pulp fibers that are calandered in a dot pattern or line pattern with generation of an embossed pattern in the compressed area and connected without a binder. Manufacture of such materials is disclosed, for example, in European patent 1 032 342.

The absorbent core can have the embossed areas on the surfaces facing the skin and/or the surfaces facing away from the skin. The fiber layer of the absorbent core is thus structured such that the pulp fibers outside of these discrete embossed areas are present in loosely placed form or only weakly adhering to each other, while in the embossed areas they are compressed with each other and are in intimate connection with the neighboring pulp fibers. With this embodiment, the use of adhesives and binding agents is completely unnecessary for formation of the composite of pulp fibers; this enables a simple and complete recycling. In the embossed areas the fibers do not simply adhere to each other. Instead, because of pressure loading, it is achieved that neighboring pulp fibers in these embossed areas are fixedly connected to each other. This connection is capable of resisting the action of moisture so that the absorbent body according to the invention is distinguished by mechanical load capacity even in the wet state.

The areas outside of the discrete embossed areas in which the fibers are present in loosely laid form or in a form where they only weakly adhere to each other are characterized by an excellent absorption capacity. The absorbed liquid is absorbed by the fibers and is distributed across the entire surface area of the absorbent core and stored therein.

In order to further increase the absorption capacity of the absorbent core, the absorbent core may contain partides of superabsorbent polymers (SAP). They can be, for example, directly worked in during manufacture of the absorbent core.

### BRIEF DESCRIPTION OF THE DRAWINGS

The present invention will be explained in more detail with the aid of the attached figures.

FIG. 1 is a plan view onto the bandage.

FIG. 2 is a section illustration along the line II-II in FIG. 1.

FIG. 3 is a perspective illustration of a section of an absorbent core.

FIG. 4 is a detail illustration of a section through the absorbent core.

FIG. 5 shows one embodiment of the widened portion of the slit end.

FIG. 6 shows a further embodiment of the widened portion in the area of the slit end.

### DESCRIPTION OF PREFERRED EMBODIMENTS

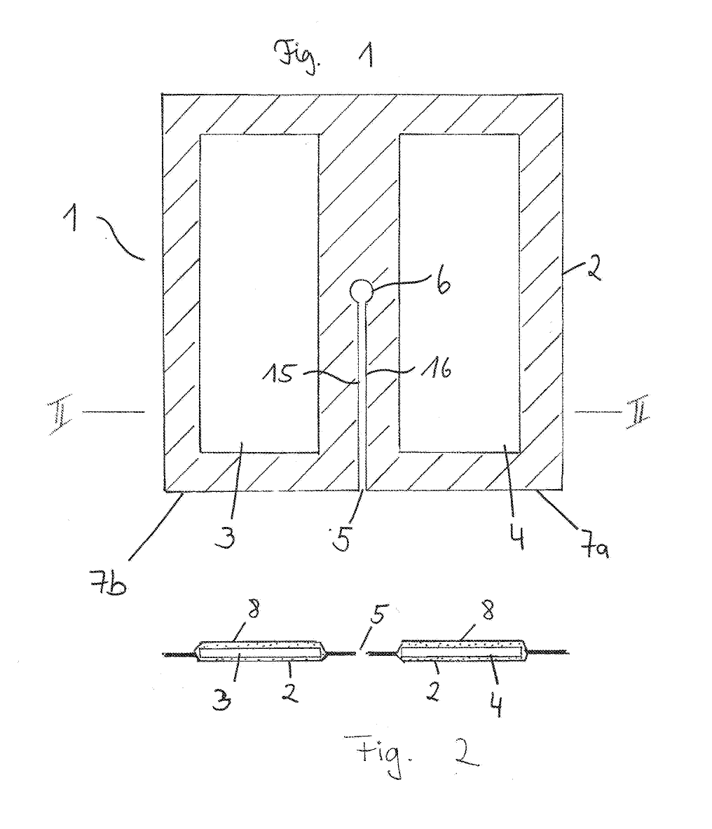

FIG. 1 shows a plan view onto the bandage according to the invention which in this embodiment is a so-called slitted compress 1. The slitted compress has a support 2 and applied thereto flat absorbent cores 3, 4. The support 2 has a slit 5 that opens into a widened portion (expansion) 6, here shown in a round (circular) shape. Because of the slit 5, a first support half 7a and a second support half 7b are defined. On each of the two support halves 7a, 7b, a flat absorbent core 3, 4 is disposed in parallel orientation to the slit 5, respectively.

The bandage according to the invention can be placed onto wounds or surgically caused body openings wherein a hose, a cannula or other line (generally referred to in this application as conduit) projecting from the wound or the body opening passes through the widened portion 6 of the slit and is surrounded by the rim of this widened portion. In this way, also the edges 15, 16 of the slit 5 are resting on the skin surface of a patient. Bodily liquid that is exiting from the body opening or wound, such as blood, ichor, orother bodily liquids, referred to, for short, as exudate, is absorbed bythe support 2 and transported in the direction toward the absorbent cores 3, 4. The absorbent cores 3, 4 absorb the liquid and store it so that rewetting in the direction toward the body surface or even to the exterior is largely avoided.

In FIG. 2, an embodiment of the bandage according to the invention is shown in section illustration along section line II-II. In this embodiment the absorbent cores 3 and 4 are covered on their respective flat sides or faces by a first support 2 and an additional (second) support 8. The support 2 and the additional support 8 have substantially the same dimensions. In areas in which the support materials project past the respective absorbent core 3, 4, the projecting portions of the supports 2, 8 are connected to each other, preferably adhesively. In the embodiment illustrated here, the absorbent cores 3, 4 each are connected adhesively, preferably by an adhesive (indicated by dots), to the support 2, 8. With this immediate connection of support and absorbent core the stability of the bandage according to the invention and also the absorption of the exiting body liquid is improved.

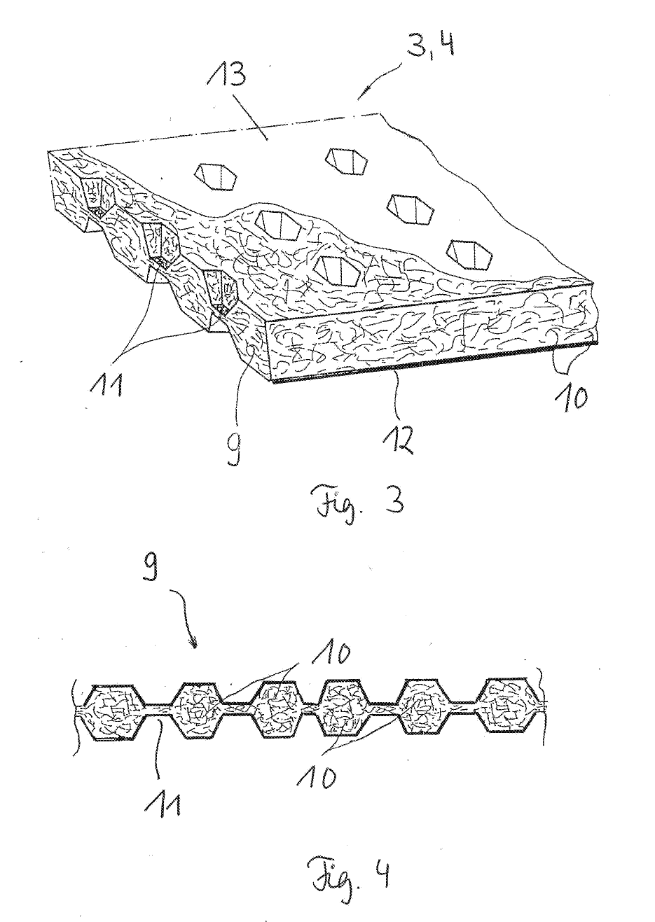

In FIG. 3 a perspective illustration of a section of an absorbent body or core 3, 4 is illustrated. In FIG. 4 a detail illustration of a possible embodiment of the absorbent core 3, 4 is illustrated in cross-section.

The layer 9 that determines the absorption behavior for liquids, in particular ichor, is preferably comprised of pulp fibers 10. The pulp fibers 10 are compressed in the embossed areas 11 and in this way connected to each other. In the embodiment illustrated here, the embossed areas 11 are located opposite each other on the top side and bottom side so that in the respective embossed area 11 only a narrow stay of interconnected pulp material remains. The other remaining areas of the layer 9 arranged between these embossed areas 11 are in the form of a loosely laid pulp layer. An intimate connection between the pulp fibers 10 does not exist in these remaining areas. The embossed areas 11 have, in the embodiment illustrated here, the shape of truncated pyramids or truncated cones, wherein the angle of the thus formed slanted sides should be between 10° and 45°.

As cellulose material for the layer 9 inexpensively available mass-produced material can be used. Preferably, if possible, unbleached so-called fluff pulp (also called wood pulp) is used that, in contrast to a bleached pulp material, is characterized by an excellent binding behavior which improves mechanical strength of the absorbent body with regard to vertical tensile forces.

When manufactured in a continuous method, the pulp web that later on forms the layer 9 is manufactured of a layered arrangement of pulp fibers of defiberized wood pulp loosely laid in an air stream and optionally interspersed absorbent materials. The pulp web of pulp fiber is obtained by layered laying of the pulp fibers. When further absorbent materials, such as SAP (superabsorbent polymer), are added, this material addition can be done also by layered laying, for example, by alternatingly laying pulp fibers, absorbent material, pulp fibers until the desired quantity of pulp fibers and optionally absorbent material has been supplied. In the embodiment illustrated in FIG. 4 of an absorbent body or core, the layer 9 of pulp fibers 10 has on its top side and/or bottom side a further layer 12, 13, respectively. In the embodiment illustrated here, the layer 12 is the base layer and the layer 13 is the cover layer. These layers 12, 13 are preferably made of a thin tissue material. This material is already incorporated upon manufacture of the absorbent core employed according to the present invention in that the pulp fibers are loosely laid onto the tissue 12 and covered by a further tissue 13. The layered arrangement of tissue and pulp as well as optionally a further tissue layer can be introduced together into the above described caland er. The tissue material is known in the prior art and can be obtained commercially as web material. The tissue material imparts additional stability to the absorbent body; it can also prevent that fine wear debris of the pulp fibers and/or of the absorbent materials can penetrate through the liquid-permeable layer facing the body in the direction toward the wound.

For manufacturing a standardized wood pulp product, it is possible to use commercially available renewable wood resources.

The method for layering pulp as a starting material for the layer 9 enables dry processing of the pulp fibers 10 and thus an excellent fusion of the pulp fibers in the discrete embossed areas by means of the subsequent embossment process between two structured embossing rollers. Outside of the embossed areas 11 the fibers are loosely resting on each other; this improves the absorption capacity and flexibility of the layer 9.

The absorption behavior of the layer 9 is substantially “dry”, i.e. as long as a complete saturation has not been reached, the absorbed liquid cannot be squeezed out as it is possible with a sponge. Even after use and as long as the absorption capacity of the absorbent layer has not been exhausted, the absorbent mat therefore appears dry.

Manufacture is done based on web material which is produced in a continuous process. In air-supported laying, first the pulp fibers and the SAPs are laid for forming the layer 9. Subsequently, in a calanderwith two structured calander rollers the production of the embossed areas 11 is realized. In further continuous steps the top layer 3 above and the bottom layer 2 below are arranged and adhesively connected with the core layer 9. The thus produced web material is subsequently cut into individual, preferably rectangular, fields that are supplied in a further manufacturing step for producing the absorbent body according to the invention.

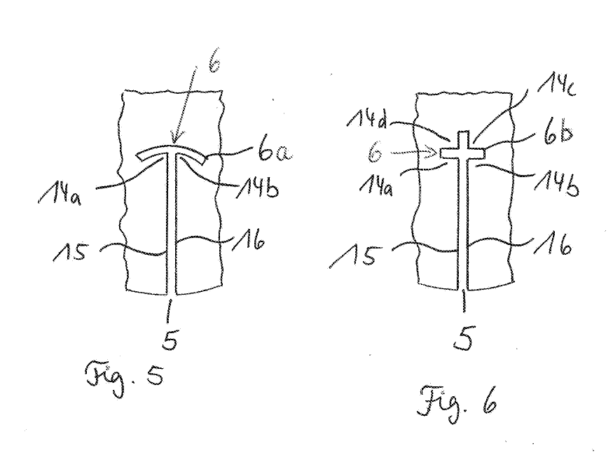

In FIGS. 5 and 6 two further possible embodiments for designing the slit in the area of its end are illustrated.

In FIG. 6 the widened portion 6 in the area of the end of the slit 5 is a transverse slit 6b that is arranged shortly before the end of the slit 5 transverse to the main slit 5. The bandage can be placed by means of the slit 5 about the conduit (cannula or hose or the like). The corners 14a, b, c, d of the bandage material that are formed by the expansion or widened portion 6 in the form of the transverse slit can bend upwardly or downwardly and thus will lie closely on the hose or the cannula such that it is held in place. The corners 14a, b, c, d formed by the transverse slit 6b and the main slit 5 bend downwardly or upwardly as required by the size of the hose or the cannula.

In the embodiment illustrated here, the bandage according to the invention can be used as a slitted compress for placement about surgically placed conduits of different size.

Moreover, it has been found that when the layers 12, 13 are of a material that is permeable for bodily liquids, liquid that is exiting from the body opening is already absorbed by the material of the corners 14a, b, c, d and the adjoining areas and is transported in the direction toward the absorbent core 3, 4 by means of the support layers.

In the embodiment illustrated in FIG. 5 the widened portion 6 opens, all the way at the end of the main slit 5, into a slit 6a which is substantially arranged transversely to the main slit 5. In the embodiment illustrated here, this transverse slit 6a describes an arc. The arc-shaped slit 6a forms with the end of the slit 5 the corners 14a, b that, as disclosed in connection with the embodiment of FIG. 6, can bend away and can thus lie closely against the hose or the cannula and secure it in place.

The slit 5 illustrated in the FIGS. 5 and 6 and the widened portion 6 in the area of the end of the slit 5 can be simple cuts. It is also possible to design the slit 5 and the widened portion 6 to be somewhat larger in that the material is stamped out in the form of the slit so that the slit edges 15, 16 and the oppositely positioned corners 14a, b, c, d, respectively, are spaced apart from each other. When the slit is a simple cut, the edges 15, 16 and the oppositely positioned corners 14a, b, c, d directly adjoin each other.

The specification incorporates by reference the entire disclosure of German priority document 20 2010 007 967.5 having a filing date of Jul. 2, 2010.

While specific embodiments of the invention have been shown and described in detail to illustrate the inventive principles, it will be understood that the invention may be embodied otherwise without departing from such principles.

What is claimed is:

1. A bandage for covering a body opening from which a conduit, not belonging to the body, projects, the bandage comprising:
a first support with a first slit beginning at an edge of the support and extending toward a center of the first support, wherein the first slit defines a first support half and a second support half opposed to each other relative to the first slit;
a first flat absorbent core disposed on the first support half;
a second flat absorbent core disposed on the second support half;
wherein the first and second absorbent cores are positioned in parallel orientation relative to the first slit, respectively;
wherein the first slit has an end remote from the edge of the first support and wherein the end of the first slit is provided with a widened portion.

2. The bandage according to claim 1, wherein the widened portion is a keyhole expansion.

3. The bandage according to claim 1, wherein the widened portion is formed by a transverse second slit extending transversely relative to the first slit.

4. The bandage according to claim 1, further comprising a second support, wherein the first and second absorbent cores each have a flat side that is facing away from the first support and wherein the second support is disposed on the flat sides facing away from the first support.

5. The bandage according to claim 4, wherein the second support and the first support have an identical shape.

6. The bandage according to claim 4, wherein at least one of the first support and the second support has a projecting portion extending past a surface of the first and second absorbent cores.

7. The bandage according to claim 4, wherein the first support and the second support have projecting portions extending past a surface of the absorbent cores and wherein the projecting portions are connected to each other.

8. The bandage according to claim 4, wherein the first and second absorbent cores are adhesively connected to the first support and the second support.

9. The bandage according to claim 1, wherein the first and second absorbent cores are adhesively connected to the first support.

10. The bandage according to claim 1, wherein the first and second absorbent cores are selected from nonwoven or woven material, which material is plastic or cellulose.

11. The bandage according to claim 10, wherein the first and second absorbent cores are comprised of an airlaid nonwoven.

12. The bandage according to daim 1, wherein the first and second absorbent cores are made of pulp fibers and are provided with embossed areas created by compression forces and distributed across a surface area of the first and second absorbent cores, respectively, wherein, in the embossed areas, the pulp fibers are more strongly compressed with each other than in remaining areas and are connected in the embossed areas by the compression forces without adhesive and/or binding agent.

13. The bandage according to claim 12, wherein the embossed areas have a truncated cone shape or a truncated pyramid shape.

14. The bandage according to claim 12, wherein the first and second absorbent cores comprise an absorbent layer containing fibers of wood pulp that are laid in an airstream and further comprise a layer of tissue above and a layer of tissue below the fibers of wood pulp.

15. The bandage according to claim 1, wherein the first and second absorbent cores contain particles of superabsorbent polymers.

16. The bandage according to claim 1, further comprising a second support arranged on a side of the first and second absorbent cores facing away from the first support, wherein the first and second supports are liquid-permeable and are comprised of nonwoven material, woven material, or punctured two-dimensional or three-dimensional film.

17. The bandage according to claim 16, wherein the first and second supports are identical.

18. The bandage according to claim 16, wherein the first and second supports are different.

19. The bandage according to claim 16, wherein the first and second absorbent cores are adhesively connected with at least one of the first support and the second support.

20. The bandage according to claim 1, wherein the first and second absorbent cores are adhesively connected with the first support.

21. The bandage according to claim 1 in the form of a slitted compress.
